(19) United States
(12) Patent Application Publication
(10) Pub. No.: US 2024/0001302 A1
(43) Pub. Date: Jan. 4, 2024

(54) METHOD AND APPARATUS FOR ENHANCED TRANSPORT

(71) Applicant: BOUNDLESS SCIENCE, LLC, Lancaster, PA (US)

(72) Inventors: Saibal BANDYOPADHYAY, Lancaster, PA (US); William F. WALKER, Lancaster, PA (US); Andrew K. JONES, Lancaster, PA (US)

(21) Appl. No.: 18/039,938

(22) PCT Filed: Dec. 1, 2021
(86) PCT No.: PCT/US2021/061447

§ 371 Date: Jun. 1, 2023

Related U.S. Application Data
(60) Provisional application No. 63/120,126, filed on Dec. 1, 2020.

(51) Int. Cl. B01D 61/24; A61M 1/16; B01D 69/08

(52) U.S. Cl. CPC B01D 61/243; B01D 69/08; A61M 1/1698; A61M 1/16

(57) ABSTRACT

A system and method for enhancing transport of matter between two media. The system includes a membrane separating the two media wherein the first media contacts at least one surface area of the membrane. Further included is a transducer configured to direct acoustic energy into the first medium proximate the at least one surface area of the membrane. In this manner, the system accelerates transport of the matter from the first to the second media.

### CROSS-REFERENCE TO RELATED APPLICATIONS

This application claims benefit of priority of U.S. Provisional Application No. 63/120,126, filed Dec. 1, 2020, which is incorporated by reference in its entirety.

### FIELD

The present application is directed to the use of acoustic energy such as ultrasound to enhance transport of matter, such as gasses or waste products, across membranes.

### BACKGROUND

Use of membranes to mediate transport of matter between media are used in a range of applications, especially medical applications. For example, the extra-corporeal membrane oxygenation (ECMO) device mediates transport of oxygen across a membrane and into blood to assist the cardiovascular system of a patient. However, such devices often rely on membranes with large surface areas increasing the need for anticoagulants to avoid fouling and adverse effects on the patient.

Thus, there remains a need for further improvements in systems for enhancing transport across membranes.

### SUMMARY

Disclosed herein is a system and method for enhancing transport of matter between two media. The system includes a membrane separating the two media wherein the first media contacts at least one surface area of the membrane. Further included is a transducer configured to direct acoustic energy into the first medium proximate the at least one surface area of the membrane. In this manner, the system accelerates transport of the matter from the first to the second media. The system is useful in dialysis and extracorporeal membrane oxygenation for example. The system also can have industrial applications, such as in the oil and gas industry.

A method of another embodiment enhances transport of a matter from a first medium across a membrane to a second medium. The method includes supplying power to at least one transducer to generate acoustic energy. Also, directing the acoustic energy into the first medium proximate at least one surface area of the membrane. The method also includes accelerating transport of the matter from the first medium through the membrane into the second medium using the acoustic energy.

Another embodiment includes dialysis specific applications. For example, the system enhances transport of waste products between blood and dialysate. The system can include a dialysis membrane and at least one transducer. The dialysis membrane is porous and separates the blood from the dialysate. The blood contacts at least one surface area of the membrane. The dialysate contacts an opposite surface area of the membrane. The at least one transducer is configured to direct acoustic energy into one of the blood or dialysate proximate the at least one surface area and the opposite surface area of the membrane. This accelerates transport of the waste products from the blood to the dialysate. Waste products can include excretable solutes such as urea, inorganic phosphate or any other small molecule with molecular weight<1 KDa.

### DESCRIPTION OF DRAWINGS

FIG. 1 is a schematic of a system for enhancing transport across a membrane of one embodiment of the present invention;

FIG. 2 is a schematic another embodiment of a system for enhancing transport with a focused energy field;

FIG. 3 is a schematic of another embodiment of a system for enhancing transport with a side-mounted transducer,

FIG. 4 is a schematic of another embodiment of a system for enhancing transport with an angled transducer;

FIG. 5 is a schematic of another embodiment of a system for enhancing transport with a top-mounted transducer directed at a second medium;

FIG. 6 is a schematic of another embodiment of a system for enhancing transport with a medium having an osmotic pressure similar to blood;

FIG. 7 is a schematic of another embodiment of a system for enhancing transport with a top-mounted transducer directed at a second medium having an osmotic pressure similar to blood;

FIG. 8 is a schematic of another embodiment of a system for enhancing transport with an acoustically active membrane;

FIG. 9 is a schematic of another embodiment of a system for enhancing transport with an acoustically active membrane and a second medium with an osmotic pressure similar to blood;

FIG. 10 is a schematic of another embodiment of a system for enhancing transport with bubbles on a porous membrane;

FIG. 11 is a schematic of another embodiment of a system for enhancing transport with bubbles within a porous membrane;

FIG. 12 is a schematic of another embodiment of a system for enhancing transport with a bubble generator; and

FIGS. 13-16 are graphs showing an increase in matter transport across a membrane using ultrasound of other embodiments of the present invention.

### DETAILED DESCRIPTION

The following description of certain examples of the inventive concepts should not be used to limit the scope of the claims. Other examples, features, aspects, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the embodiments of this disclosure are described herein. The described methods, systems, and apparatus should not be construed as limiting in any way. Instead, the present disclosure is directed toward all novel and nonobvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect, embodiment or example of the invention are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

Further, the terms “coupled” and “associated” generally means electrically, electromagnetically, and/or physically (e.g., mechanically or chemically) coupled or linked and does not exclude the presence of intermediate elements between the coupled or associated items.

“Optional” or “optionally” means that the subsequently described event or circumstance may or may not occur, and that the description includes instances where said event or circumstance occurs and instances where it does not.

The term “transducer” refers to a device that converts energy of one type to energy of another type. In the present disclosure, the term transducer will generally refer to a device that converts electrical energy to ultrasound energy. In portions of the present disclosure, we will use the terms “acoustic source,” “ultrasound source,” and “ultrasound emitter” to be generally synonymous with the term transducer. Ultrasound transducers may include piezoelectric ceramics (including lead-ziconate-titanate), piezoelectric polymers (including PVDF), MEMS transducers, CMUTs, PMUTs, PolyMUTs, and other technologies The term ultrasound refers to acoustic energy with frequencies above approximately 20 KHz. Unless specifically noted, the term “fluid” will refer to blood, dialysate, or other fluids of practical interest, including non-biological fluids such as petroleum.

One embodiment of the present invention applies ultrasound energy to enhance diffusion of gas through a semi-permeable membrane. In one embodiment, a membrane separates a gas-filled chamber from a fluid-filled chamber. Ultrasound energy is applied to the fluid-filled chamber and is directed towards the membrane.

Without being wed to theory, the inventors have determined that ultrasound energy enhances diffusion through a number of mechanisms. First, whenever an ultrasound wave propagates through a medium with finite attenuation (practically all media) then the loss of ultrasound energy leads to a momentum transfer from the propagating ultrasound wave to the medium. This momentum transfer results in a local force being applied to the propagation medium. This force is localized within the propagating ultrasound field with a direction coincident with the direction of the propagating ultrasound field. This force field, known as acoustic radiation force, may cause displacements of solid media and may induce flow within fluid media. Such radiation force induced flow is known as acoustic streaming. Acoustic streaming may act to increase the transport of gases by causing flow near the membrane separating the gas and fluid. This flow may shrink the boundary layer near the membrane and therefore steepen the concentration gradient of the dissolved gas within the fluid. Because this concentration gradient drives diffusion, diffusion may increase.

The inventors have also determined that at high ultrasound intensities, non-linear propagation may cause the development of harmonic frequencies in the propagated ultrasound wave field. Likely, higher frequencies are more rapidly attenuated, so the acoustic streaming may grow more rapidly with increasing ultrasound amplitude. The improvements in transport may thus grow faster than linearly with increasing acoustic amplitude.

The improvements in transport are greater with focused ultrasound fields than with a broad, uniform ultrasound field. One reason may be that the acoustic radiation force field is localized within the ultrasound field. A uniform wave field yields a uniform force within the fluid. This bulk force does not allow for recirculation and therefore does not yield significant velocities. Higher flow velocities may disrupt the boundary layer more and may therefore yield more significant improvements in transport.

In one embodiment of the present invention, the ultrasound wavefield is generated by an unfocused transducer with dimensions selected so that the near-field/far-field transition of the wave field is located near the membrane. In this scenario the transducer “auto-focuses” so that the acoustic intensity and therefore radiation force field is localized in or before the membrane. This may yield higher flow velocities near the membrane. In other embodiments, it is considered to place the near-field/far-field transition at a distance before the membrane so that the flow field may be greater at the membrane surface.

In another embodiment of the present invention, it is advantageous to use physically focused transducers (ultrasound emitters) to localize the acoustic field and therefore the acoustic radiation force. This may allow for placement of the ultrasound focus at a distance that would not be compatible with an auto-focused system. This may be particularly valuable when it is desired to place the focus at a range near to the transducer. Such a configuration can allow for a shallower fluid layer and therefore a smaller apparatus.

In embodiments with a focused system, it may be desired to present a flat surface to the fluid, rather than a convex surface. In these embodiments, it may be desired to encapsulate the transducer to yield a desired surface geometry. One of skill in the art, studying this disclosure of the invention, will realize that unless the encapsulant has the same speed of sound as the working fluid, it may be necessary to account for speed of sound differences to ensure proper focusing of the acoustic field.

In another embodiment, it may be advantageous to use a phased array focusing system, rather than a physically focused transducer. This approach may allow a flat transducer to be focused at an arbitrary depth. A phased array system has the further advantage of being able to employ apodization to increase the depth of field of the focal field. A phased array system also allows the acoustic field (acoustic beam) to be steered. Such steering may be advantageous. In one instance the steering may be applied to direct the acoustic beam against the direction of flow of the fluid. In one such approach the applied fluid flow would be parallel to the membrane and the ultrasound beam would be directed at 45° relative to the membrane, but with a component pushing against the fluid flow direction. This approach may cause mixing of the fluid. In another instance the beam may be steered at 45° relative to the direction of flow, but along the direction of flow to accelerate the fluid. This may create a higher flow velocity and may act to collapse the boundary condition.

The embodiments of the present invention may be further supplemented by the use of an array of transducers designed to create an array of acoustic beams. Such a configuration may create multiple acoustic streaming paths. This would increase diffusion at multiple locations on the membrane simultaneously. One simple approach to create multiple beams would be to create a checkerboard of transducers with white squares being active transducers and black squares being inactive regions. Alternative geometries such as hexagonal grids, triangular grids, or other geometries would also work well.

Diffusion is a rate limited process. Ultrasound can be turned on and off very quickly. It may be advantageous to alternate ultrasound between two or more transducers. In one embodiment, the checkerboard pattern described above could alternate between a period of transmission on the white squares and a second period of transmission from the black squares.

In each of the above embodiments, it could be advantageous to coat the transducer or transducer encapsulant with material to reduce the likelihood of clot formation.

Blood (and other fluids) can be damaged by excessive temperatures. For this reason, it may be advantageous to apply active cooling to the back of the transducer. Alternatively, or in addition, the transducer can be placed within a thermally conductive fixture. As another alternative or option, the fluid itself can be cooled. The encapsulant described above can be selected to provide thermal insulation. One possible material for encapsulation is polymethylpentene (trade name TPX). This material provides an excellent acoustic match to blood (and other working fluids) and has a low thermal conductivity.

In addition to bulk streaming, resulting from acoustic radiation force within fluid, microstreaming is also a possibility. Microstreaming is the result of expansion and contraction of bubbles within a fluid. Since liquids are almost entirely incompressible, but gases are highly compressible, the application of ultrasound to a bubble may cause that bubble to expand and contract dramatically. Since the fluid may mostly retain its volume, the fluid will rush into and out of the volume around the bubble. This effect is called microstreaming which may advantageously improve oxygen transport.

The embodiments of the present invention may incorporate bubbles on the membrane in order to provide a locus for microstreaming. Microstreaming can effectively mix fluids at the membrane surface and therefore disrupt the boundary layer and enhance transport. Similar effects can be achieved by embedding bubbles within the membrane. Bubbles can also be injected within the fluid in order to enable microstreaming and associated mixing throughout the bulk of the fluid. The bubbles will also be displaced strongly by the acoustic wave field and may therefore increase streaming and associated mixing.

Absorption of the propagating ultrasound causes local heating of the fluid and the membrane if it impinges upon it. Differential heating will induce convection, which can drive an increase in transport.

In the present disclosure, the term hollow fiber membrane (HFM) refers to a hollow tube with porous walls. The HFM wall, for example, can separate the blood chamber from the fluid or gas chamber. (An HFM can be used to separate other media, including non-biological media such as petroleum and water for example.) In the embodiments of the present disclosure where HFM allows diffusion of gases into the blood, the HFM is permeable to gases. In the current embodiment of the present disclosure where HFM allows diffusion of water-soluble molecules from the blood, the HFM is permeable to water.

In one embodiment where HFM is used for diffusing gases into the blood, the inner diameter of the HFM ranges between 100μ-200μ, 200μ-300μ, 300μ-400μ, 400μ-500μ, 500μ-700μ and wall thickness ranges from 10-30μ, 30-60μ, 60-90μ and the size of the pores in the wall ranges from 1 nm, 1 nm to 3 nm, 3 nm to 5 nm, 5 nm to 10 nm, 10 nm to 100 nm, 100 nm to 500 nm, 500 nm to 1 micrometer.

In the embodiment where HFM is used for diffusing gases into the blood, the gas is passed through the hollow lumen and blood is passed over the HFM outer surface.

In one embodiment where HFM is derived from poly methyl pentene (PMP) by a melt extrusion process, the PMP HFM has an outer layer which partially covers the pores present in the wall and resists fluid from the blood to infiltrate into the hollow lumen.

In another embodiment, HFM is derived from polypropylene.

In another embodiment, expanded polytetrafluoroethylene (ePTFE) is used as HFM In this embodiment ePTFE HFM is produced by expanding PTFE tubes by forcefully pulling it from both ends.

In the embodiment where ePTFE is used as HFM, the density of ePTFE varies from 0.3-0.5 g/cc, 0.5-0.7 g/cc, 0.7-0.9 g/cc, 0.9-1.2 g/cc, 1.2-1.5g/cc, 1.5-1.8 g/cc, 1.8-2.0 g/cc.

In another embodiment of the current disclosure, the HFM comprises polyvinylidene fluoride (PVDF) which has piezoelectric property.

In another embodiment of the current disclosure, the HFM comprises a porous ceramic which has piezoelectric property. The porous ceramic HFM can be produced by casting a composite film of ceramic particle dispersion and a polymer binder which undergoes thermal degradation during the subsequent sintering step.

In another embodiment of the current disclosure, the HFM is made out of PVDF and ceramic composite which has piezoelectric property.

In an embodiment of the present disclosure where HFM is used for the diffusion of gases into the blood, the blood contacting surface is coated with an antithrombogenic coating which includes, but is not limited to phosphatidyl choline, heparin, or bovine serum albumin.

In an embodiment of the present disclosure where the HFM is used for diffusing excretable solutes from the blood into a secondary fluid, the inner diameter of the HFM varies between 100μ-200μ, 200μ-300μ, 300μ-400μ, wall thickness varies between 10-20μ, 20-30μ, 30-40μ, 40-50μ, 50-70μ and pore size varies between 1-3 nm, 3-5nm, 5-10 nm, 10-20 nm, 20-50 nm.

In an embodiment of the present disclosure where the HFM is used for diffusing excretable solutes from the blood into a secondary fluid, the blood is passed through the hollow fiber and the secondary fluid of identical osmotic pressure of blood is passed over the HFM. Excretable solutes diffused and convected through the HFM are removed by the secondary fluid

In an embodiment of the present disclosure where the HFM is used for diffusing excretable solutes from the blood into a secondary fluid, the HFM can comprise polysulfone or polyether sulfone or polyvinyl pyrrolidone or sulfonated polyacrylonitrile or polymethylmethacrylate.

In an embodiment of the present disclosure where the HFM is used for diffusing excretable solutes from the blood into a secondary fluid, the pore size of HFM wall allows small molecules and macromolecules (<11 KDa) to diffuse through the membrane while limiting albumin leakage.

In an embodiment where ultrasound is focused on the HFM, the deposition of any protein membrane on the HFM wall is inhibited by the ultrasound.

In an embodiment where ultrasound is focused on the HFM, the diffusion rate of solutes is increased by the local heating created by the ultrasound on the HFM surface.

In an embodiment where HFM is used for diffusing excretable solutes from the blood into a secondary fluid, the biocompatibility of the membrane can be improved or supplemented by incorporating oligomeric surface-active additives into the bulk polymer during the HFM manufacturing process.

In an embodiment where HFM is used for diffusing excretable solutes from the blood into a secondary fluid, the surface charge (zeta potential) of the HFM is partially neutralized by incorporating zwitterionic surface active additives into the bulk polymer during the HFM manufacturing process.

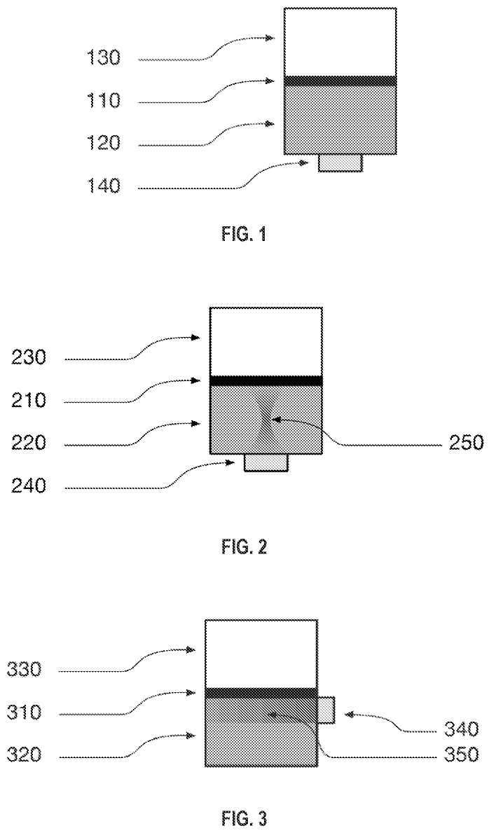

FIGS. 1-12 show various embodiments of transducers and membranes to enhance transport across the membranes and to test the same for various practical applications. FIG. 1, for example, shows a dual compartment setup with a porous membrane 110 dividing a blood compartment 120 from a gas compartment 130. An ultrasound source 140, such as an ultrasound transmitter, is positioned adjacent to the blood compartment 120. The ultrasound transmitter 140 is oriented to emit a signal generally perpendicular (generally orthogonal) to the surface of the porous membrane 110.

FIG. 2 illustrates, in another embodiment, a focused ultrasound energy field 250 emitted by a perpendicularly oriented ultrasound source 240. Other components of the system of FIG. 2 are similarly configured as FIG. 1, including a porous membrane 210 and a blood compartment 220 and a gas compartment 230 separated by the porous membrane.

FIG. 3 illustrates, in another embodiment, a porous membrane 310 separating a blood compartment 320 from a gas compartment 330. An ultrasound source 340 is positioned adjacent to the blood compartment 320 and is oriented to emit an ultrasound energy field 350 that parallels the blood-facing surface of the porous membrane 310.

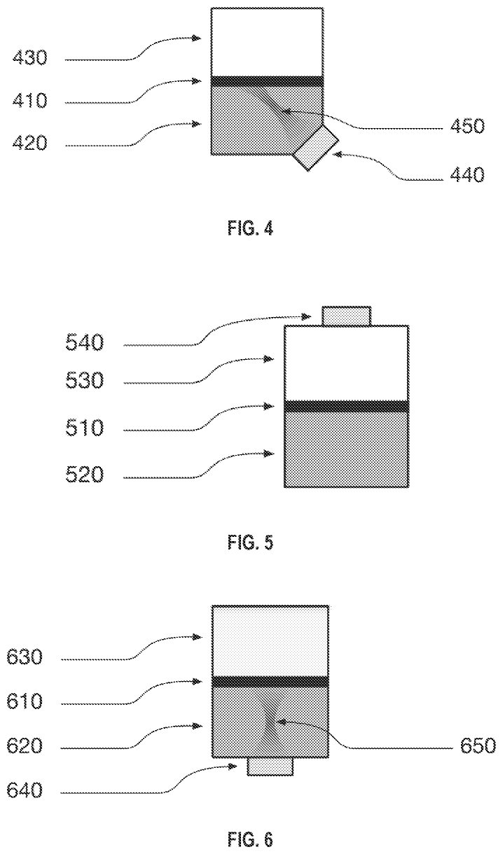

FIG. 4 illustrates, in another embodiment, a porous membrane 410 separating a blood compartment 420 from a gas compartment 430. An ultrasound source 440 is oriented at adjacent the blood compartment 420 and is oriented to emit an ultrasound energy field 450 at an angle to the surface of the porous membrane.

FIG. 5 illustrates another embodiment wherein a porous membrane 510 again separates a blood compartment 520 from a gas compartment 530, but an acoustic source 540 is positioned adjacent to the gas compartment. The acoustic source 540 is oriented generally perpendicular or orthogonal to the surface of the porous membrane 510.

FIG. 6 illustrates another embodiment wherein a porous membrane 610 separates a blood compartment 620 from a liquid compartment 630. The liquid in the liquid compartment has an osmotic pressure similar to that of blood. Like FIG. 2, an ultrasound source 640 is oriented and emits an ultrasound energy field 650 perpendicular to the surface of the porous membrane 610.

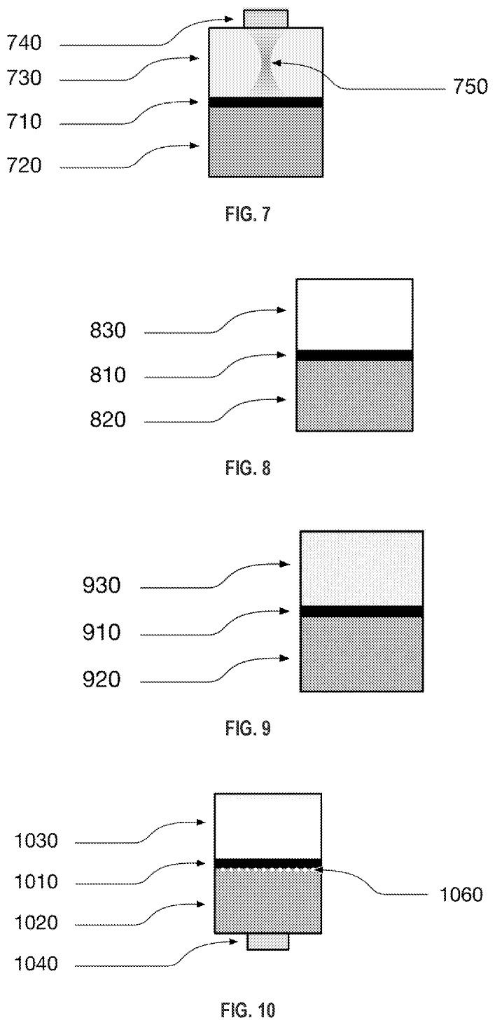

FIG. 7 illustrates another embodiment wherein a porous membrane 710 separates a blood compartment 720 from a liquid compartment 730. An ultrasound source 740 is positioned adjacent the liquid compartment 730. The ultrasound source is positioned and oriented (configured) to emit an ultrasound energy field 750 perpendicular, through a liquid with an osmotic pressure similar to blood, to the porous membrane 710.

FIG. 8 shows an embodiment wherein a porous membrane 810 separates a blood compartment 820 from gas compartment 830. In this embodiment, the porous membrane 810 is itself acoustically active.

FIG. 9 shows an embodiment wherein an acoustically active porous membrane 910 separates a blood compartment 920 from a liquid compartment 930. The liquid has an osmotic pressure similar to blood.

FIG. 10 shows an embodiment wherein a porous membrane 1010 separates a blood compartment 1020 from a gas compartment 1030. An ultrasound source 1040 is positioned adjacent the blood compartment opposite the porous membrane 1010. Also, a plurality of bubbles 1060 are positioned over the blood-side surface of the porous membrane 1010 in the pathway of an ultrasound energy field.

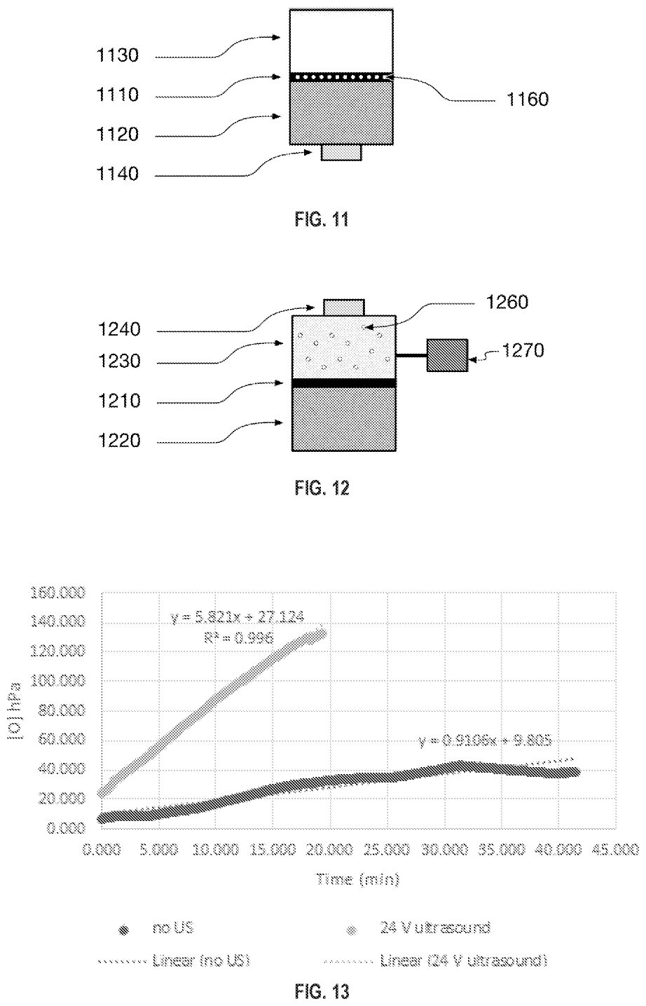

FIG. 11 shows an embodiment wherein a porous membrane 1110 separates a blood compartment 1120 from a gas compartment 1130. An ultrasound source 1140 is positioned opposite the porous membrane 1110. A plurality of bubbles 1160 are positioned within the porous membrane 1110

FIG. 12 shows an embodiment wherein a porous membrane 1201 separating a blood compartment 1220 from a liquid compartment 1230. An ultrasound source 1240 is positioned adjacent the liquid compartment 1230 and opposite the porous membrane 1201. The liquid in the liquid compartment 1230 has an osmotic pressure similar to blood. (Liquid compartments disclosed herein may have other liquids and do not necessarily require a matching of osmotic pressure with blood.) A bubble injector 1270 is positioned in fluid communication with the liquid compartment and is configured to inject a plurality of bubbles 1260 within the liquid

Exemplary testing results are provided herein for the purposes of illustration and not limitation.

Test 1: Perfluorodecalin oxygenation through a membrane as shown in FIG. 13.

In a closed loop, 60 mL perfluorodecalin (PFD) was circulated at 150 ml/min through the transducer compartment of the dual compartment setup shown in FIG. 1. The other compartment was placed under an oxygen atmosphere maintaining 3 cm hydrostatic pressure. Both compartments were separated by a semipermeable polypropylene membrane (100 nm pore size). Rate of PFD oxygenation was measured real time by recording the dissolved oxygen concentration using PreSens OXY-4 SMA oximeter probe working on an oxidative photobleaching mechanism. The rate of oxygenation was compared between two separate experiments run in the presence and absence of ultrasound. FIG. 13 shows the effect of ultrasound on polypropylene membrane mediated oxygenation of perfluorodecalin. The rate of oxygenation increased by 6.3 fold under the influence of 24V ultrasound focused on polypropylene membrane.

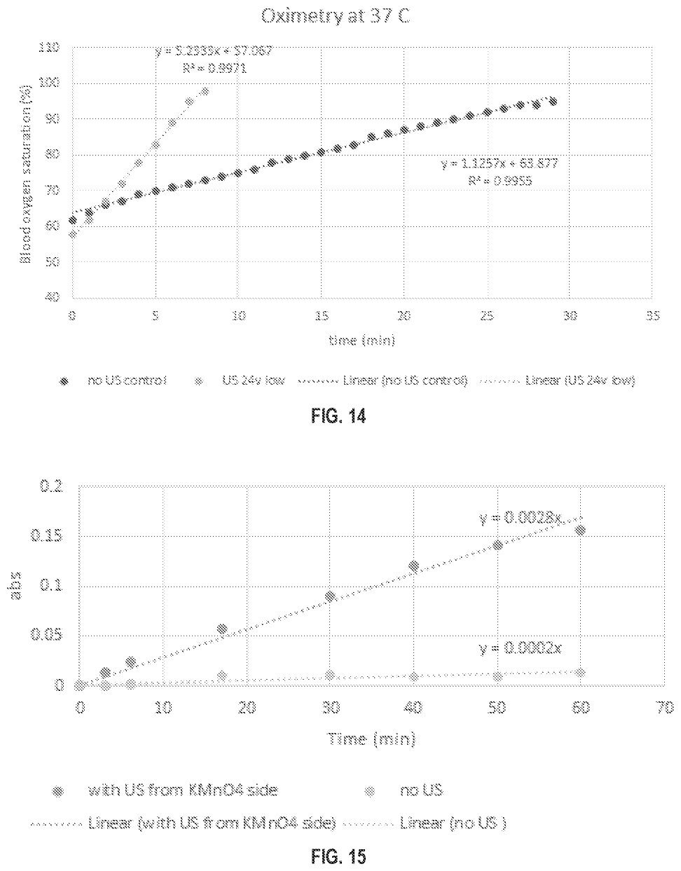

Test 2: Whole blood oxygenation through a membrane as shown in FIG. 14.

In a closed loop, 60 mL whole blood was circulated at 150 ml/min through the transducer compartment of a dual compartment setup shown in FIG. 1. The other compartment was placed under an oxygen atmosphere maintaining 3 cm hydrostatic pressure. Both compartments were separated by a semipermeable polypropylene membrane (100 nm pore size). The rate of whole blood oxygenation was measured real time by recording the blood oxygen saturation using Fresenius CRIT LINE III pulse oximeter probe. The rate of oxygenation was compared between two separate experiments run in the presence and absence of ultrasound. FIG. 14 shows effect of ultrasound on polypropylene membrane mediated oxygenation of whole blood at 37 C. The rate of oxygenation increased by 4.6 fold under the influence of 24V ultrasound focused on polypropylene membrane.

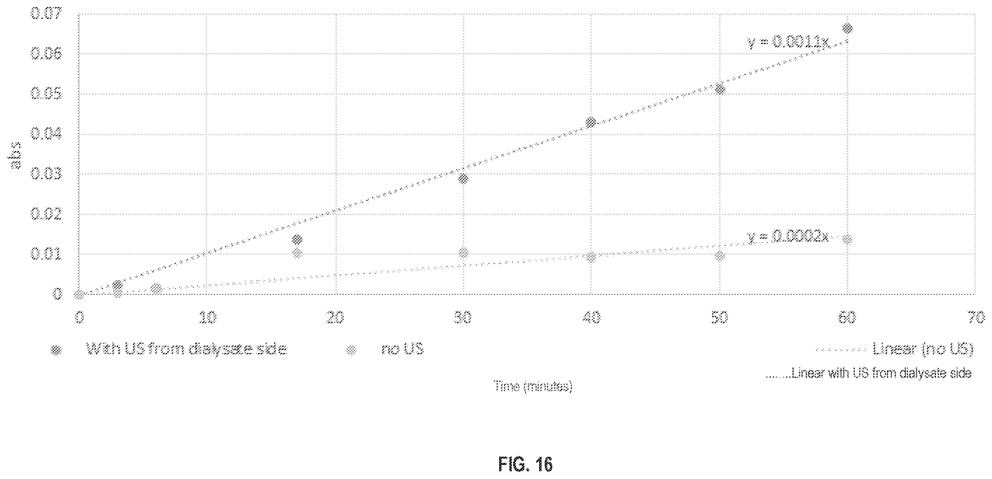

Test 3: Whole blood removal of small molecules through a membrane as shown in FIGS. 15 and 16.

In a closed loop, 20 mL KMnO4 solution in phosphate buffer saline (PBS) was placed in the transducer compartment of a dual compartment setup shown in FIG. 1. 200 mL PBS buffer as dialysate was circulated through the other compartment at 150 ml/min flow rate. Both compartments are separated by a semipermeable polysulfone membrane (30 nm pore size). The rate of KMnO4 diffusion was measured by collecting 300 uL dialysate solution at every time point. The rate of KMnO4 diffusion was compared between two separate experiments run in the presence and absence of ultrasound. This experiment was repeated with the transducer placed both in the KMnO4 side and the dialysate side.

FIG. 15 shows improvement of KMnO4 diffusion rate by 14 fold when a 24 v ultrasound transducer is placed in the KMnO4 solution side and the beam was focused on the membrane. The KMnO4 concentration in flowing dialysate was measured at different time points by UV-vis absorption spectroscopy at 562 nm wavelength. FIG. 16 shows improvement of KMnO4 diffusion rate by 5.5 fold when a 24 v ultrasound transducer is placed in the dialysate side and the beam was focused on the membrane. The KMnO4 concentration in flowing dialysate was measured at different time points by UV-vis absorption spectroscopy at 562 nm wavelength.

### Exemplary Aspects

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: An apparatus for diffusing gases into blood comprising a porous membrane, blood on one side of said porous membrane, a gas on the other side of said membrane, and an ultrasound source configured to direct ultrasound energy into the blood, aimed toward said membrane.

Example 2: The apparatus according to any example herein, particularly example 1, further comprising a membrane with pore size between 0 to 1 nm, 1 nm to 3 nm, 3 nm to 5 nm, 5 nm to 10 nm, 10 nm to 100 nm, 100 nm to 500 nm, or 500 nm to 1 micrometer.

Example 3: The apparatus according to any example herein, particularly example 1, comprising a membrane made out of polypropylene (PP), or polymethyl pentene (PMP) or polytetrafluoroethylene (PTFE) or polyvinylidene fluoride (PVDF) or a composite made out of polyvinylidene fluoride and inorganic oxides.

Example 4: The apparatus according to any example herein, particularly example 1, further comprising of a hollow fiber membrane (HFM) with lumen size 10-50μ, 50-100μ, 100-200μ, 200-300μ, 300-400μ, 400-500μ, 500-600μ, or 600-700μ.

Example 5: The apparatus according to any example herein, particularly example 1, further comprising of a plurality of HFMs bundled together.

Example 6: The apparatus according to any example herein, particularly example 1, further comprising of an ultrasound source device which creates sound waves between 20 KHz to 100 KHz, 100 KHz to 1 MHz, 1 MHz to 10 MHz, or 10 MHz to 100 MHz.

Example 7: The apparatus according to any example herein, particularly example 1, further comprising an ultrasound source configured to direct ultrasound energy along a direction perpendicular to said membrane.

Example 8: The apparatus according to any example herein, particularly example 1, further comprising an ultrasound source configured to direct ultrasound energy along a direction tangential to said membrane.

Example 9: The apparatus according to any example herein, particularly example 1, further comprising an ultrasound source configured to direct ultrasound energy along a direction at an angle between parallel and perpendicular to said membrane.

Example 10: The apparatus according to any example herein, particularly example 1, where the gas is oxygen.

Example 11: The apparatus according to any example herein, particularly example 1, where the gas is a combination of oxygen and nitrogen.

Example 12: The apparatus according to any example herein, particularly example 1, where the gas is a combination of several gasses, which can include oxygen, nitrogen, carbon dioxide, argon, nitric oxide, and any gases commonly found in air.

Example 13: An apparatus for enhanced diffusion of gases into blood comprising a porous membrane, blood on one side of said porous membrane, a gas on the other side of said membrane, and an acoustic device placed in the gas in the vicinity of the porous membrane.

Example 14: The apparatus according to any example herein, particularly example 13, further comprising a membrane with pore size between 0 to 1 nm, 1 nm to 3 nm, 3 nm to 5 nm, 5 nm to 10 nm, 10 nm to 100 nm, 100 nm to 500 nm, or 500 nm to 1 micrometer.

Example 15: The apparatus according to any example herein, particularly example 13, comprising a membrane made out of polypropylene (PP), or polymethyl pentene (PMP) or polytetrafluoroethylene (PTFE) or polyvinylidene fluoride (PVDF) or a composite made out of polyvinylidene fluoride and inorganic oxides.

Example 16: The apparatus according to any example herein, particularly example 13, further comprising of a hollow fiber membrane (HFM) with lumen size 10-50μ, 50-100μ, 100-200μ, 200-300μ, 300-400μ, 400-500μ, 500-600μ, or 600-700μ.

Example 17: The apparatus according to any example herein, particularly example 13, further comprising of a plurality of HFMs bundled together.

Example 18: The apparatus according to any example herein, particularly example 13, further comprising of an acoustic device which creates sound waves between 0 KHz to 100 KHz, 100 KHz to 1 MHz, 1 MHz to 10 MHz, or 10 MHz to 100 MHz.

Example 19: The apparatus according to any example herein, particularly example 13, further comprising an ultrasound source configured to direct ultrasound energy along a direction perpendicular to said membrane.

Example 20: The apparatus according to any example herein, particularly example 13, further comprising an ultrasound source configured to direct ultrasound energy along a direction parallel to said membrane.

Example 21: The apparatus according to any example herein, particularly example 13, further comprising an ultrasound source configured to direct ultrasound energy along a direction at an angle between parallel and perpendicular to said membrane.

Example 22: An apparatus for the diffusion of excretable solutes in blood into a secondary solution comprising a porous membrane, blood on one side of said porous membrane, a liquid with osmotic pressure similar to blood on the other side of said membrane and an ultrasound source configured to direct ultrasound energy toward said membrane.

Example 23: The apparatus according to any example herein, particularly example 22, further comprising a membrane with pore size between 0 to 1 nm, 1 nm to 3 nm, 3 nm to 5 nm, 5 nm to 10 nm, or 10 nm to 100 nm.

Example 24: The apparatus according to any example herein, particularly example 22, comprising a membrane made out of polysulfone, or polyether sulfone or polymethyl methacrylate or sulfonated polyacrylonitrile.

Example 25: The apparatus according to any example herein, particularly example 22, further comprising of a hollow fiber membrane (HFM).

Example 26. The apparatus according to any example herein, particularly example 22, further comprising of a plurality of HFMs formed into a bundle.

Example 27: The apparatus according to any example herein, particularly example 22, further comprising of an acoustic device which generates ultrasound between 20 KHz to 100 KHz, 100 KHz to 1 MHz, 1 MHz to 10 MHz, or 10 MHz to 100 MHz.

Example 28: The apparatus according to any example herein, particularly example 22, further comprising an ultrasound source configured to direct ultrasound energy along a direction perpendicular to said membrane.

Example 29: The apparatus according to any example herein, particularly example 22, further comprising an ultrasound source configured to direct ultrasound energy along a direction parallel to said membrane.

Example 30: The apparatus according to any example herein, particularly example 22, further comprising an ultrasound source configured to direct ultrasound energy along a direction at an angle between parallel and perpendicular to said membrane.

Example 31: The apparatus according to any example herein, particularly example 22, wherein the ultrasound source is placed in the blood on the blood side of said membrane.

Example 32: The apparatus according to any example herein, particularly example 22, wherein the ultrasound source is placed in the liquid side (not the blood side) of said membrane.

Example 33: The apparatus according to any example herein, particularly example 22, where the non-blood liquid is dialysate.

Example 34: A method of improving the gas transfer through a porous membrane into blood comprising the use of ultrasound energy passed through the blood.

Example 35: The method according to any example herein, particularly example 34, where the ultrasound energy in the blood is between 0 KHz to 100 KHz, 100 KHz to 1 MHz, 1 MHz to 10 MHz, or 10 MHz to 100 MHz.

Example 36: The method according to any example herein, particularly example 34, where the ultrasound energy is directed from the blood towards the membrane.

Example 37: The method according to any example herein, particularly example 34, where the ultrasound energy is directed in the blood tangentially to the membrane.

Example 38: A method of improving gas transfer through a porous membrane into blood comprising the application of acoustic energy through a gas on the gas side of the membrane.

Example 39: The method according to any example herein, particularly example 38, where the acoustic energy is directed at the membrane.

Example 40: The method according to any example herein, particularly example 34, where the acoustic energy in the gas is between 1 Hz to 100 KHz, 100 KHz to 1 MHz, 1 MHz to 10 MHz, or 10 MHz to 100 MHz.

Example 41: An apparatus for the enhancing the diffusion of gases into blood comprising, an acoustically active porous membrane, blood on one side of said porous membrane, a gas on the other side of said membrane.

Example 42: An apparatus for the diffusion of excretable solutes in blood into a secondary solution comprising an acoustically active porous membrane, blood on one side of said porous membrane, and a liquid with osmotic pressure similar to blood on the other side of said membrane.

Example 43: An apparatus for the diffusion of excretable solutes in blood into a secondary solution comprising a porous membrane with gas bubbles adhered to its surface or embedded within it, blood on one side of said porous membrane, a liquid with osmotic pressure similar to blood on the other side of said membrane, and an ultrasound source configured to direct ultrasound energy towards said membrane.

Example 44. An apparatus for diffusing gases into blood comprising, a porous membrane with gas bubbles adhered to its surface or embedded within it, blood on one side of said porous membrane, a gas on the other side of said membrane and an ultrasound source configured to direct ultrasound energy into the blood.

Example 45: The apparatus according to any example herein, particularly examples 43 or 44, wherein said bubbles are anchored to a pattern of hydrophobic regions on said membrane.

Example 46: The apparatus according to any example herein, particularly examples 43 or 44, wherein said bubbles are encapsulated and said encapsulated bubbles are anchored to said membrane.

Example 47: The apparatus according to any example herein, particularly examples 43 or 44, wherein said bubbles are encapsulated within said membrane.

Example 48: An apparatus for diffusing gases into blood comprising a porous membrane, blood on one side of said porous membrane, a gas on the other side of said membrane, a bubble injector that injects bubbles into said blood.

Example 49: An apparatus for the diffusion of excretable solutes in blood into a secondary solution comprising a porous membrane, blood on one side of said porous membrane, a liquid with osmotic pressure similar to blood on the other side of said membrane, an ultrasound source configured to direct ultrasound energy within said blood and/or said liquid, a bubble injector that injects bubbles into said liquid and/or said blood.

Example 50: The apparatus according to any example herein, particularly examples 1 or 13 or 34 or 38 or 41 or 44 or 48, wherein the membrane is naturally hydrophobic and coated with a biocompatible coating at least in the blood contacting side.

Example 51, The apparatus according to any example herein, particularly example 50, wherein the biocompatible coating is either immobilized heparin, protein, phosphocholine functionalized lipids or polymers, or polyethylene glycol.

Example 52: The apparatus according to any example herein, particularly examples 22 or 42 or 43 or 49 wherein the membrane is hydrophilic by nature or coated with a hydrophilic coating to improve its biocompatibility.

Example 53: The apparatus according to any example herein, particularly example 52, wherein the biocompatible coating on the membrane includes poly-N-vinylpyrrolidinone (PVP), polyethylene co-vinyl alcohol and polyethylene glycol, poly sulfobetaine methacrylate, or sulfobetaine silane.

Example 54: The apparatus according to any example herein, particularly example 52, wherein the biocompatible coating partially neutralizes the zeta potential of porous membrane allowing highly negatively charged solutes, such as inorganic phosphates, to pass through.

Example 55: An apparatus for the diffusion of excretable solutes in blood into a secondary solution comprising a porous membrane, blood on one side of said porous membrane, a liquid with osmotic pressure similar to blood on the other side of said membrane, an ultrasound source and an optional acoustic device.

Example 56: The apparatus according to any example herein, particularly example 55, wherein the excretable solute includes urea, inorganic phosphate or any other small molecule with molecular weight<1 KDa.

Example 57. The apparatus according to any example herein, particularly example 55, wherein the excretable solute includes β-microglobulin or any macromolecule with molecular weight ranging between 1-5 KDa, 5-10 KDa, or 10-15 KDa.

Example 58: The apparatus according to any example herein, particularly example 55, wherein the porous membrane does not allow macromolecules of >59KDa molecular weight to pass through.

Example 59: The apparatus according to any example herein, particularly example 55, wherein the membrane allows water to remove 1-10 ml/h/mmHg/m2, 10-20 ml/h/mmHg/m2, 20-40 ml/h/mmHg/m2, or 40-80 ml/h/mmHg/m2.

Example 60: A system for enhancing transport of a matter between a first medium and a second medium, the system comprising a membrane separating the first medium from the second media wherein the first media contacts at least one surface area of the membrane, and

at least one transducer configured to direct acoustic energy into the first medium proximate the at least one surface area of the membrane to accelerate transport of the matter from the first media into the second media.

Example 61: The system according to any example herein, particularly example 60, wherein the first medium has a higher concentration of the matter than the second medium.

Example 62: The system according to any example herein, particularly example 60, wherein the second medium has a higher concentration of the matter than the first medium.

Example 63: The system according to any example herein, particularly examples 60 or 61, wherein the first medium is blood and wherein the matter includes oxygen.

Example 64: The system according to any example herein, particularly example 60, wherein the matter transport is enhanced by at least about twofold

Example 65: The system according to any example herein, particularly example 60, wherein the matter transport is enhanced by at least about one of threefold, fourfold, fivefold or sixfold.

Example 66: The system according to any example herein, particularly example 60, further comprising a source of bubbles coupled to at least one of the first media or second media and configured to inject a plurality of bubbles thereinto.

Example 67: The system according to any example herein, particularly example 60, wherein the acoustic energy has a frequency of at least 20 KHz.

Example 68. The system according to any example herein, particularly example 60, wherein the at least one transducer includes a focusing mechanism configured to focus the acoustic energy toward the at least one surface area.

Example 69: The system according to any example herein, particularly example 60, wherein the at least one surface area of the membrane is free of interfering deposits from the first medium.

Example 70 The system according to any example herein, particularly example 60, wherein the at least one transducer is configured to generate a near-field and far-field transition near the at least one surface area of the membrane.

Example 71: The system according to any example herein, particularly example 70, wherein the at least one transducer is configured to auto-focus the near-field and far field transition.

Example 72: The system according to any example herein, particularly example 60, wherein the at least one transducer is physically focused to localize the acoustic field near to the transducer.

Example 73: The system according to any example herein, particularly example 60, wherein the at least one transducer is configured to present a flat surface to the first medium.

Example 74: The system according to any example herein, particularly example 60, wherein the at least one transducer includes a phased array focusing system.

Example 75: The system according to any example herein, particularly example 74, wherein the phased array focusing system is configured to steer the acoustic energy.

Example 76: The system according to any example herein, particularly example 60, wherein the at least one transducer includes a plurality of transducers in a checkerboard pattern.

Example 77: The system according to any example herein, particularly example 76, wherein white transducers of the checkerboard pattern are configured to alternate with black transducers of the checkboard pattern in directing acoustic energy.

Example 78: The system according to any example herein, particularly example 60, wherein the at least one transducer includes an encapsulant acoustically matched to the first medium.

Example 79: The system according to any example herein, particularly example 60, further comprising a thermal management system associated with the transducer

Example 80: The system according to any example herein, particularly example 60, wherein the membrane is associated with a plurality of bubbles.

Example 81: The system according to any example herein, particularly example 80, wherein the bubbles are contained within the membrane.

Example 82: The system according to any example herein, particularly example 60, wherein the membrane is a hollow fiber membrane.

Example 83: The system according to any example herein, particularly example 82, wherein the at least one transducer is further configured to inhibit deposition of proteins on the hollow fiber membrane via directed acoustic energy.

Example 84: A method of enhancing transport of a matter from a first medium across a membrane to a second medium, the method comprising supplying power to at least one transducer to generate acoustic energy, directing the acoustic energy into the first medium proximate at least one surface area of the membrane, and accelerating transport of the matter from the first medium through the membrane into the second medium using the acoustic energy.

Example 85: The method according to any example herein, particularly example 84, wherein accelerating transport includes accelerating transport by at least one of about twofold, threefold, fourfold, fivefold or sixfold.

Example 86: The method according to any example herein, particularly example 85, further comprising injecting a plurality of bubbles into the first medium.

Example 87: The method according to any example herein, particularly example 84, including focusing the acoustic energy toward the at least one surface area.

Example 88: The method according to any example herein, , particularly example 84, further comprising directing acoustic energy to prevent interfering deposits on the membrane.

Example 89: The method according to any example herein, particularly example 84, further comprising generating a near field and far-field transition near the at least one surface area of the membrane.

Example 90: The method according to any example herein, particularly example 84, wherein the at least one transducer includes a plurality of transducers arranged in a checkerboard pattern and further comprising alternating white transducers with black transducers.

Example 91. The method according to any example herein, particularly example 84, further comprising managing a temperature of the first medium.

Example 92: The method according to any example herein, particularly example 84, wherein the one of the first or second media includes a dialysate and the matter includes a waste product.

Example 93: The system according to any example herein, particularly example 60, wherein the one of the first or second media includes a dialysate and the matter includes a waste product.

Example 94: The system according to any example herein, particularly example 60, wherein the at least one transducer includes an acoustically active component of the membrane.

Example 95: A system for enhancing transport of waste products between blood and dialysate, the system comprising a dialysis membrane separating the blood from the dialysate wherein the blood contacts at least one surface area of the membrane and the dialysate contacts an opposite surface area of the membrane, and at least one transducer configured to direct acoustic energy into one of the blood or dialysate proximate the at least one surface area and the opposite surface area of the membrane to accelerate transport of the waste products from the blood into the dialysate.

Example 96. The system according to any example herein, particularly example 95, wherein the waste products are excretable solutes.

Example 97: The system according to any example herein, particularly example 96, wherein the excretable solute includes urea, inorganic phosphate or any other small molecule with molecular weight<1 KDa.

Example 98: The system according to any example herein, particularly example 95, wherein the dialysate has an osmotic pressure similar to the blood.

Example 99: The system according to any example herein, particularly example 95, further comprising a bubble injector configured to inject bubbles into one of the blood or the dialysate.

Example 100: The system according to any example herein, particularly example 95, wherein the waste products transport is enhanced by at least about one of twofold, threefold, fourfold, fivefold or sixfold.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove and that many modifications and other embodiments are intended to be included within the scope of the appended claims. Moreover, although specific terms are employed herein, as well as in the claims which follow, they are used only in a generic and descriptive sense and not for the purposes of limiting the described invention nor the claims which follow. We, therefore, claim as our invention all that comes within the scope and spirit of these claims.

1. A system for enhancing transport of a matter between a first medium and a second medium, the system comprising:
a membrane separating the first medium from the second media wherein the first media contacts at least one surface area of the membrane; and
at least one transducer configured to direct acoustic energy into the first medium proximate the at least one surface area of the membrane to accelerate transport of the matter from the first media into the second media.

2. The system of claim 1, wherein the first medium has a higher concentration of the matter than the second medium.

3. The system of claim 1, wherein the second medium has a higher concentration of the matter than the first medium.

4. The system of claim 1, wherein the first medium is blood and wherein the matter includes oxygen.

5. The system of claim 1, wherein the matter transport is enhanced by at least about twofold.

6. The system of any one of the preceding claims claim 1, wherein the matter transport is enhanced by at least about one of threefold, fourfold, fivefold or sixfold.

7. The system of claim 1, further comprising a source of bubbles coupled to at least one of the first media or second media and configured to inject a plurality of bubbles thereinto.

8. The system of claim 1, wherein the acoustic energy has a frequency of at least 20 KHz.

9. The system of claim 1, wherein the at least one transducer includes a focusing mechanism configured to focus the acoustic energy toward the at least one surface area.

10. The system of claim 1, wherein the at least one surface area of the membrane is free of interfering deposits from the first medium.

11. The system of claim 1, wherein the at least one transducer is configured to generate a near-field and far-field transition near the at least one surface area of the membrane.

12. The system of claim 11, wherein the at least one transducer is configured to auto-focus the near-field and far field transition.

13. The system of claim 1, wherein the at least one transducer is physically focused to localize the acoustic field near to the transducer.

14. The system of claim 1, wherein the at least one transducer is configured to present a flat surface to the first medium.

15. The system of claim 1, wherein the at least one transducer includes a phased array focusing system.

16. The system of claim 15, wherein the phased array focusing system is configured to steer the acoustic energy.

17. The system of claim 1, wherein the at least one transducer includes a plurality of transducers in a checkerboard pattern.

18. The system of claim 17, wherein white transducers of the checkerboard pattern are configured to alternate with black transducers of the checkboard pattern in directing acoustic energy.

19. The system of claim 1, wherein the at least one transducer includes an encapsulant acoustically matched to the first medium.

20. The system of claim 1, further comprising a thermal management system associated with the transducer.

21. The system of claim 1, wherein the membrane is associated with a plurality of bubbles.

22. The system of claim 21, wherein the bubbles are contained within the membrane.

23. The system of claim 1, wherein the membrane is a hollow fiber membrane.

24. The system of claim 23, wherein the at least one transducer is further configured to inhibit deposition of proteins on the hollow fiber membrane via directed acoustic energy.

25. A method of enhancing transport of a matter from a first medium across a membrane to a second medium, the method comprising:
supplying power to at least one transducer to generate acoustic energy;
directing the acoustic energy into the first medium proximate at least one surface area of the membrane; and
accelerating transport of the matter from the first medium through the membrane into the second medium using the acoustic energy.

26. The method of claim 25, wherein accelerating transport includes accelerating transport by at least one of about twofold, threefold, fourfold, fivefold or sixfold.

27. The method of claim 26, further comprising injecting a plurality of bubbles into the first medium.

28. The method of claim 25, including focusing the acoustic energy toward the at least one surface area.

29. The method of claim 25, further comprising directing acoustic energy to prevent interfering deposits on the membrane.

30. The method of claim 25, further comprising generating a near field and far-field transition near the at least one surface area of the membrane.

31. The method of claim 25, wherein the at least one transducer includes a plurality of transducers arranged in a checkerboard pattern and further comprising alternating white transducers with black transducers.

32. The method of claim 25, further comprising managing a temperature of the first medium.

33. The method of claim 25, wherein the one of the first or second media includes a dialysate and the matter includes a waste product.

34. The system of claim 1, wherein the one of the first or second media includes a dialysate and the matter includes a waste product.

35. The system of claim 1, wherein the at least one transducer includes an acoustically active component of the membrane.

36. A system for enhancing transport of waste products between blood and dialysate, the system comprising:
a dialysis membrane separating the blood from the dialysate wherein the blood contacts at least one surface area of the membrane and the dialysate contacts an opposite surface area of the membrane; and
at least one transducer configured to direct acoustic energy into one of the blood or dialysate proximate the at least one surface area and the opposite surface area of the membrane to accelerate transport of the waste products from the blood into the dialysate.

37. A system of claim 36, wherein the waste products are excretable solutes.

38. A system of claim 37, wherein the excretable solute includes urea, inorganic phosphate or any other small molecule with molecular weight<1 KDa.

39. A system of claim 36, wherein the dialysate has an osmotic pressure similar to the blood.

40. A system of claim 36, further comprising a bubble injector configured to inject bubbles into one of the blood or the dialysate.

41. A system of claim 36, wherein the waste products transport is enhanced by at least about one of twofold, threefold, fourfold, fivefold or sixfold.
